(19) United States
(12) Patent Application Publication
(10) Pub. No.: US 2021/0283058 A1
(43) Pub. Date: Sep. 16, 2021

(54) METHOD FOR PRODUCING SUSTAINED-RELEASE DRUG, AND SUSTAINED-RELEASE DRUG

(71) Applicant: GUNZE LIMITED

(72) Inventor: Hidetaka NAKAYAMA, Kyoto (JP)

(21) Appl. No.: 16/334,587

(22) PCT Filed: Aug. 31, 2017
(86) PCT No.: PCT/JP2017/031410

§ 371 Date: Mar. 19, 2019

(30) Foreign Application Priority Data
Sep. 21, 2016 (JP) 2016-184314

(51) Int. Cl. A61K 9/16; A61K 9/19; A61K 31/727

(52) U.S. Cl. CPC A61K 9/1682; A61K 9/19; B82Y 5/00; A61K 9/1647; A61K 31/727

(57) ABSTRACT

The present invention aims to provide a method for producing a sustained-release drug which includes allowing a medicinal ingredient to be supported on a porous body containing a bioabsorbable polymer and which can control the releasing rate of the medicinal ingredient. The present invention also aims to provide a sustained-release drug. The present invention is directed to a method for producing a sustained-release drug, including: a solution preparing step of preparing a medicinal ingredient-bioabsorbable polymer solution having a medicinal ingredient uniformly dispersed therein and a bioabsorbable polymer dissolved therein, using the bioabsorbable polymer, the medicinal ingredient, a solvent 1 that is a poor solvent having a lower solvency for the bioabsorbable polymer, a solvent 2 that is a good solvent having a higher solvency for the bioabsorbable polymer and is incompatible with the solvent 1, and a common solvent 3 compatible with the solvent 1 and the solvent 2; a precipitating step of cooling the medicinal ingredient-bioabsorbable polymer solution to precipitate a porous body containing the bioabsorbable polymer and the medicinal ingredient; and a freeze-drying step of freeze-drying the porous body containing the bioabsorbable polymer and the medicinal ingredient to provide a sustained-release drug containing a porous body supporting the medicinal ingredient, wherein one or two or more common solvents 3 are used, and a releasing rate of the medicinal ingredient of the resulting sustained-release drug is controlled by adjusting a type and a mixing ratio of the one or two or more common solvents 3.

### TECHNICAL FIELD

The present invention relates to a method for producing a sustained-release drug which includes allowing a medicinal ingredient to be supported on a porous body containing a bioabsorbable polymer and which can control the releasing rate of the medicinal ingredient. The present invention also relates to a sustained-release drug.

### BACKGROUND ART

Oral administration is a conventionally known method for administering a drug to a patient. The orally administered drug immediately disintegrates to release its active ingredient, thus quickly exhibiting effects. Meanwhile, sustained-release drugs have been proposed. Sustained-release drugs are specially designed to slowly release their medicinal ingredients. Since sustained-release drugs release their medicinal ingredients little by little over a long time, they can reduce the number of doses or prevent severe adverse effects.

A known method for controlling the sustained release of the medicinal ingredient is to control the releasing rate by allowing the medicinal ingredient to be supported on a porous body containing a bioabsorbable polymer (see Patent Literatures 1 and 2, for example).

Sustained-release drugs produced using porous bodies containing bioabsorbable polymers are promising for applications in regenerative medicine. Regenerative medicine is an attempt to reconstruct human tissues or organs using various animal cells, including human cells. For example, the most frequently used artificial blood vessels in clinic are those containing non-absorbable polymers, such as GORE-TEX. Unfortunately, artificial blood vessels containing non-absorbable polymers remain as foreign matter in the body for a long time after grafting, so that anticoagulants and the like have to be continuously administered. In addition, when such artificial blood vessels are used in children, repeat surgery is disadvantageously required as they grow older. To overcome the situation, regeneration of blood vessel tissue using porous bodies containing bioabsorbable polymers has been attempted.

Prevention of thrombus formation is also important for the regeneration of blood vessel tissue by regenerative medicine. Thrombus formation often causes clogging of blood vessels, particularly in regeneration of small-diameter blood vessel tissue. Such clogging not only prevents regeneration of normal blood vessels, but also may cause severer symptoms. In such a situation, if an anticoagulant such as heparin can be incorporated in a porous body containing a bioabsorbable polymer and the heparin slowly released with decomposition of the porous body, thrombus formation can be prevented for a long time without continuous administration of an anticoagulant. For example, Non-Patent Literature 1 discloses such a porous body containing a bioabsorbable polymer and heparin. Specifically, Non-Patent Literature 1 discloses a method for producing a nanofiber material for blood vessels, wherein an aqueous heparin sodium solution and a surfactant are added to an organic solvent having a bioabsorbable polymer dissolved therein, and the resulting micellar solution is used to produce the nanofiber material.

The required releasing rate of the medicinal ingredient varies depending on the purpose or stage. For example, when heparin is released from a porous body containing a bioabsorbable polymer in regeneration of blood vessel tissue by regenerative medicine, a comparatively large amount of heparin needs to be released for the post-grafting period up to about 24 to 72 hours when acute thrombosis is a concern. Thereafter, the sustained release of heparin needs to be relatively small and constant. Unfortunately, with conventional sustained-release drugs, it is difficult to control the releasing rate of the medical ingredient according to the purpose or stage.

### CITATION LIST

### Patent Literature

Patent Literature 1: JP H08-175981 A
Patent Literature 2: JP 2007-520614 T

### Non-Patent Literature

Non-Patent Literature 1: Colloids and Surfaces B: Biointerfaces, 128 (2015), 106-114

### SUMMARY OF INVENTION

### Technical Problem

In view of the situation in the art, the present invention aims to provide a method for producing a sustained-release drug which includes allowing a medicinal ingredient to be supported on a porous body containing a bioabsorbable polymer and which can control the releasing rate of the medicinal ingredient. The present invention also aims to provide a sustained-release drug.

### Solution to Problem

The present invention is directed to a method for producing a sustained-release drug, including: a solution preparing step of preparing a medicinal ingredient-bioabsorbable polymer solution having a medicinal ingredient uniformly dispersed therein and a bioabsorbable polymer dissolved therein, using the bioabsorbable polymer, the medicinal ingredient, a solvent 1 that is a poor solvent having a lower solvency for the bioabsorbable polymer, a solvent 2 that is a good solvent having a higher solvency for the bioabsorbable polymer and is incompatible with the solvent 1, and a common solvent 3 compatible with the solvent 1 and the solvent 2; a precipitating step of cooling the medicinal ingredient-bioabsorbable polymer solution to precipitate a porous body containing the bioabsorbable polymer and the medicinal ingredient; and a freeze-drying step of freeze-drying the porous body containing the bioabsorbable polymer and the medicinal ingredient to provide a sustained-release drug containing a porous body supporting the medicinal ingredient, wherein one or two or more common solvents 3 are used, and a releasing rate of the medicinal ingredient of the resulting sustained-release drug is controlled by adjusting a type and a mixing ratio of the one or two or more common solvents 3.

The present invention is described in detail below.

For porous bodies containing bioabsorbable polymers, control of their properties such as pore size and bulk density is extremely important from the standpoint of mechanical strength as a tissue regeneration scaffold, bioabsorption behavior, cell permeability, supply of nutrition to cells entering the porous body, and the like. A known process for producing such a porous body containing a bioabsorbable polymer is the phase separation process, which includes forming a homogenous phase by mixing a good solvent and a poor solvent for the bioabsorbable polymer, followed by cooling to give a porous body. In the phase separation process, the pore size of the resulting porous body can be adjusted by adjustment of the mixing ratio between the good solvent and the poor solvent. However, the adjustment of the pore size of the porous body in the phase separation process greatly changes the bulk density of the resulting porous body. Specifically, for production of a porous body having a large pore size, the ratio of the poor solvent has to be high. This makes the ratio of the good solvent relatively low, so that the resulting porous body has a high bulk density. Conversely, for production of a porous body having a small pore size, the ratio of the good solvent is set high and that of the poor solvent set low, so that the resulting porous body has a low bulk density. It is thus very difficult to produce a porous body having a different pore size but the same bulk density by the phase separation process. Furthermore, the phase separation process requires that the good solvent and the poor solvent are compatible with each other. When water, which is easy to handle, is selected as the poor solvent, there are only limited choices of good solvents such as 1,4-dioxane, N-methylpyrrolidone, and dimethyl sulfoxide.

These solvents are highly toxic to the living body, and thus a step for completely removing the solvents from the porous body is required for clinical application. This disadvantageously makes the production process complicated.

The present inventors made intensive studies to devise a method for producing a porous body in which good and poor solvents for a bioabsorbable polymer are used in combination with a common solvent compatible with both of the good and poor solvents. With the common solvent, the good solvent and the poor solvent do not have to be compatible with each other. This allows a much wider choice of combinations of good solvents and poor solvents. In addition, in the production method, a less toxic organic solvent other than 1,4-dioxane, N-methylpyrrolidone, or dimethyl sulfoxide can be selected as the good solvent. Furthermore, the bulk density and pore size of the porous body can be easily adjusted by combining two or more common solvents and adjusting the mixing ratio between the two or more common solvents.

The present inventors found out that a sustained-release drug containing a porous body supporting a medicinal ingredient can be produced by selecting a solvent capable of dissolving the medicinal ingredient as the good solvent or the poor solvent and dissolving the medicinal ingredient in the good solvent or the poor solvent. The present inventors further found out that the releasing rate of the medicinal ingredient of the sustained-release drug can be controlled by using one or two or more common solvents and adjusting the type and mixing ratio of the one or two or more common solvents. They thus completed the present invention.

In the method for producing a sustained-release drug of the present invention, first, a solution preparing step is performed. In this step, a medicinal ingredient-bioabsorbable polymer solution having a medicinal ingredient uniformly dispersed therein and a bioabsorbable polymer dissolved therein is prepared using the bioabsorbable polymer, the medicinal ingredient, a solvent 1 that is a poor solvent having a lower solvency for the bioabsorbable polymer, a solvent 2 that is a good solvent having a higher solvency for the bioabsorbable polymer and is incompatible with the solvent 1, and a common solvent 3 compatible with the solvent 1 and the solvent 2.

Examples of the bioabsorbable polymer include synthetic polymers such as polyglycolide, polylactide, poly-ε-caprolactone, lactide-glycolic acid copolymer, glycolide-€-caprolactone copolymer, lactide-ε-caprolactone copolymer, polycitric acid, polymalic acid, poly-α-cyanoacrylate, poly-β-hydroxy acid, polytrimethylene oxalate, polytetramethylene oxalate, polyorthoester, polyorthocarbonate, polyethylene carbonate, poly-γ-benzyl-L-glutamate, poly-γ-methyl-L-glutamate, poly-L-alanine, polyglycol, and sebacic acid, polysaccharides such as starch, alginic acid, hyaluronic acid, chitin, pectic acid, and derivatives thereof, and natural polymers such as proteins (e.g., gelatin, collagen, albumin, fibrin). These bioabsorbable materials may be used alone or in combination of two or more thereof.

The medicinal ingredient may be either a water-soluble drug or a poorly water-soluble drug (hydrophobic drug and fat-soluble drug). A water-soluble drug and a poorly water-soluble drug may be used in combination.

The “water-soluble drug” herein means a drug having high solubility in water. Specifically, for example, “water-soluble drug” means that the drug has solubility in water corresponding to, according to the terms of The Japanese Pharmacopoeia, Seventeenth Edition, “Very soluble (the volume of the solvent required for dissolving 1 g or 1 mL of the solute is less than 1 mL)”, “Freely soluble (the volume of the solvent required for dissolving 1 g or 1 mL of the solute is from 1 mL to less than 10 mL)”, “Soluble (the volume of the solvent required for dissolving 1 g or 1 mL of the solute is from 10 mL to less than 30 mL)”, or “Sparingly soluble (the volume of the solvent required for dissolving 1 g or 1 mL of the solute is from 30 mL to less than 100 mL)”.

The “poorly water-soluble drug” herein means a drug having low solubility in water. Specifically, for example, the “poorly water-soluble drug” means that the drug has solubility in water corresponding to, according to the terms of The Japanese Pharmacopoeia, Seventeenth Edition, “Practically insoluble, or insoluble (the volume of the solvent required for dissolving 1 g or 1 mL of the solute is 10000 mL and over)”, “Very slightly soluble (the volume of the solvent required for dissolving 1 g or 1 mL of the solute is from 1000 mL to less than 10000 mL)”, or “Slightly soluble (the volume of the solvent required for dissolving 1 g or 1 mL of the solute is from 100 mL to less than 1000 mL)”.

Examples of the water-soluble drug include antithrombotic drugs such as heparin, aspirin, acenocoumarol, phenindione, and EDTA, vitamins such as sodium ascorbate and B vitamins, amino acids such as glutamic acid, aspartic acid, and taurine, sugars such as oligosaccharides, galactose, and trehalose, and antibiotics such as β-lactam antibiotics, aminoglycoside antibiotics, DPT, LZD, and colistin. These water-soluble drugs may be used alone or in combination of two or more thereof.

Examples of the poorly water-soluble drug include common additives such as L-menthol and olive oil, fat-soluble vitamins such as vitamin E and vitamin A, antithrombotic drugs such as warfarin, antibiotics such as avermectin, ivermectin, spiramycin, and ceftiofur, antibacterial agents such as amoxicillin, erythromycin, oxytetracycline, and lincomycin, antiinflammatory agents such as dexamethasone and phenylbutazone, hormones such as levothyroxine, adrenocortical steroids such as dexamethasone palmitate, triamcinolone acetonide, and halopredone acetate, nonsteroidal antiinflammatory drugs such as indometacin and aspirin, therapeutic agents for arterial occlusion such as prostaglandin El, antitumor drugs such as actinomycin and daunomycin, drugs for diabetes such as acetohexamide, and therapeutic agents for bone disease such as estradiol. These poorly water-soluble drugs may be used alone or in combination of two or more thereof.

The solvent 1 is a poor solvent having a lower solvency for the bioabsorbable polymer. The “poor solvent” as used herein means that the solvent is less likely to dissolve the bioabsorbable polymer than the solvent 2 is, and more specifically means that the mass of the bioabsorbable polymer that dissolves in 100 g of the solvent 1 at a room temperature of 25° C. is 0.01 g or less.

In cases where the bioabsorbable polymer is a synthetic polymer, the solvent 1 may be water, methanol, n-propanol, isopropanol, or n-butanol, for example. In particular, water is suitable because it has excellent handleability.

The solvent 2 is a good solvent having a higher solvency for the bioabsorbable polymer. The “good solvent” as used herein means that the solvent is more likely to dissolve the bioabsorbable polymer than the solvent 1 is, and more specifically means that the mass of the bioabsorbable polymer that dissolves in 100 g of the solvent 2 at a room temperature of 25° C. is 0.1 g or more.

The solvent 2 is incompatible with the solvent 1. The “incompatible” as used herein means that phase separation occurs even after mixing and stirring at a room temperature of 25° C.

In cases where the bioabsorbable polymer is a synthetic polymer and water is selected as the solvent 1, the solvent 2 may be an organic solvent such as methyl ethyl ketone, diethyl ketone, methyl propyl ketone, methyl isobutyl ketone, methylamino ketone, cyclohexanone, chloroform, ethyl acetate, or toluene. In particular, for example, methyl ethyl ketone and chloroform are suitable because they have relatively low toxicity.

The common solvent 3 is compatible with both of the solvent 1 and the solvent 2. Combining such a common solvent 3 with the solvent 1 and the solvent 2 enables production of a porous body by the phase separation process even if the solvent 1 and the solvent 2 are incompatible with each other. This remarkably widens the choice of combinations of the solvents 1 and 2. The “compatible” as used herein means that phase separation does not occur even after mixing and stirring at a room temperature of 25° C.

The pore size of the resulting porous body can be controlled by adjusting the mixing ratio between the solvent 1 and the solvent 2. Specifically, an increase in the ratio of the solvent 1 increases the pore size of the resulting porous body, and an increase in the ratio of the solvent 2 decreases the pore size of the porous body.

The mixing ratio between the solvent 1 and the solvent 2 is not limited. Preferably, the weight ratio between the solvent 1 and the solvent 2 is within the range of 1:1 to 1:100. With the weight ratio within this range, a uniform porous body can be produced. The weight ratio is more preferably within the range of 1:10 to 1:50.

The mixing ratio between the total of the solvent 1 and the solvent 2 and the common solvent 3 is not limited. Preferably, the weight ratio between the total of the solvent 1 and the solvent 2 and the common solvent 3 is within the range of 1:0.01 to 1:0.5. With the weight ratio within this range, a uniform porous body can be produced. The weight ratio is more preferably within the range of 1:0.02 to 1:0.3.

In cases where the bioabsorbable polymer is a synthetic polymer and water and an organic solvent are selected as the solvent 1 and the solvent 2 respectively, the common solvent 3 may be, for example, acetone, methanol, ethanol, propanol, isopropanol, n-butanol, 2-butanol, isobutanol, or tetrahydrofuran.

In the method for producing a sustained-release drug of the present invention, one or two or more common solvents 3 are used, and the releasing rate of the medicinal ingredient of the resulting sustained-release drug is controlled by adjusting the type and the mixing ratio of the one or two or more common solvents 3 (hereinafter, the two or more solvents included in the common solvent 3 are also referred to as a “common solvent 3-1”, a “common solvent 3-2”, . . . ). Although the mechanism is not entirely clear, the releasing rate can be controlled presumably because, for example, the adjustment of the mixing ratio between the common solvent 3-1 and the common solvent 3-2 changes the micelle size of the dispersed medicinal ingredient, thus changing the size of the medicinal ingredient exposed on the wall of the resulting porous body.

The pore size of the resulting porous body can be controlled by combining two or more common solvents 3, for example, a common solvent 3-1 and a common solvent 3-2, and adjusting the mixing ratio between these solvents. In other words, the pore size of the resulting porous body also can be controlled by adjusting the mixing ratio between the common solvent 3-1 and the common solvent 3-2 included in the common solvent 3 while holding the mixing ratio between the solvent 1, the solvent 2, and the common solvent 3 constant. This means that the bulk density of the resulting porous body can remain substantially constant while only the pore size is adjusted. This method for producing a sustained-release drug of the present invention makes it easy to produce a sustained-release drug containing a porous body having desired pore size and bulk density.

The combination of the bioabsorbable polymer and the solvents is not limited. Examples of the combination include: a combination of a lactide-ε-caprolactone copolymer as the bioabsorbable polymer with water as the solvent 1 and methyl ethyl ketone as the solvent 2; a combination of polylactide as the bioabsorbable polymer with water as the solvent 1 and chloroform as the solvent 2; and a combination of polylactide as the bioabsorbable polymer with water as the solvent 1 and chloroform as the solvent 2. These combinations each may be further combined with ethanol as the common solvent 3, or may be combined with ethanol as the common solvent 3-1 and propanol as the common solvent 3-2 at various mixing ratios.

In the solution preparing step, a medicinal ingredient-bioabsorbable polymer solution having the medicinal ingredient uniformly dispersed therein and the bioabsorbable polymer dissolved therein is prepared using the bioabsorbable polymer, the medicinal ingredient, the solvent 1, the solvent 2, and the common solvent 3.

Specific examples of the method for preparing the medicinal ingredient-bioabsorbable polymer solution include: a method involving dissolving the medicinal ingredient in the solvent 1 or the solvent 2, mixing the bioabsorbable polymer with a solvent mixture (hereinafter also referred to simply as a “solvent mixture”) containing the solvent 1, the solvent 2, and the common solvent 3, followed by heating. Simpler methods for preparing the medicinal ingredient-bioabsorbable polymer solution include: a method involving heating the solvent mixture and adding the bioabsorbable polymer to the heated solvent mixture; and a method involving dissolving the bioabsorbable polymer in the solvent 2 and then adding the solvent 1 and the common solvent 3 to the solvent 2 with heating. Even simpler methods for preparing the medicinal ingredient-bioabsorbable polymer solution include a method involving converting the medicinal ingredient into a colloid (micelles) using the solvent 1, the solvent 2, the common solvent 3, or the solvent mixture, and then adding, with heating, the colloid (micelles) of the medicinal ingredient to the solution having the bioabsorbable polymer dissolved in the solvent 2.

The mixing method is not limited. For example, a known mixing method using stirrer chips or stirring bars may be used.

In the above step, the medicinal ingredient may be dissolved in the solvent 1, the solvent 2, and the common solvent 3, whichever is capable of dissolving the medicinal ingredient. The medicinal ingredient may be converted into a colloid (micelles) using the solvent 1, the solvent 2, the common solvent 3, or the solvent mixture. For example, when the medicinal ingredient is heparin, water may be selected as the solvent 1, and heparin is dissolved therein. Furthermore, after heparin is dissolved in water as the solvent 1, ethanol as the common solvent 3 may be added to the solution to convert heparin into a colloid (micelles).

The obtained medicinal ingredient-bioabsorbable polymer solution has the bioabsorbable polymer uniformly dissolved therein and the medicinal ingredient uniformly dispersed therein. The medicinal ingredient in the medicinal ingredient-bioabsorbable polymer solution seemingly forms stable micelles by self-micellization.

The heating temperature in the solution preparing step may be any temperature at which the bioabsorbable polymer is uniformly dissolved. Preferably, the heating temperature is lower than the boiling point of any of the solvent 1, the solvent 2, and the common solvent 3. Heating to the boiling point or higher may change the mixing ratio between the solvents, which may make it impossible to control the pore size and bulk density of the resulting porous body.

In the method for producing a sustained-release drug of the present invention, next, a precipitating step is performed. In this step, the medicinal ingredient-bioabsorbable polymer solution is cooled to precipitate a porous body containing the bioabsorbable polymer and the medicinal ingredient. Cooling the medicinal ingredient-bioabsorbable polymer solution precipitates a porous body containing the bioabsorbable polymer that has become insoluble. This is presumably because, before the bioabsorbable polymer crystallizes and precipitates, phase separation (liquid-liquid phase separation) of the bioabsorbable polymer in the liquid state and the solvents occurs due to thermodynamic instability at a temperature higher than the temperature at which the bioabsorbable polymer crystallizes. At this time, the medicinal ingredient dispersed in the medicinal ingredient-bioabsorbable polymer solution uniformly adheres, by van der Waals forces or the like, to the surface of the precipitated porous body containing the bioabsorbable polymer.

The cooling temperature in the precipitating step may be any temperature at which the porous body containing the bioabsorbable polymer can precipitate. Preferably, the temperature is 4° C. or lower, more preferably −24° C. or lower.

The cooling rate also affects the pore size of the resulting porous body. Specifically, a higher cooling rate tends to result in a smaller pore size, and a slower cooling rate tends to result in a larger pore size. Thus, especially for production of a porous body having a small pore size, the cooling temperature may be set low to rapidly cool the medicinal ingredient-bioabsorbable polymer solution.

In the method for producing a sustained-release drug of the present invention, next, a freeze-drying step is performed. In this step, the obtained porous body containing the bioabsorbable polymer and the medicinal ingredient is freeze-dried to give a sustained-release drug containing a porous body supporting the medicinal ingredient.

The freeze-drying may be performed under any conditions, and may be performed under conventionally known conditions.

The freeze-drying step may be performed after the cooling step without any further treatment; however, for removal of organic solvents used as the solvents, the porous body may be immersed in a solvent such as ethanol to replace the organic solvents before freeze-drying. The solvent used at this time is one that does not dissolve the medicinal ingredient so that the medicinal ingredient does not dissolve out of the porous body.

According to the method for producing a sustained-release drug of the present invention, a sustained-release drug containing a porous body supporting a medicinal ingredient can be produced in a very simple manner. In particular, the releasing rate of the medicinal ingredient of the resulting sustained-release drug can be controlled by using one or two or more common solvents 3 and adjusting the type and mixing ratio of the one or more common solvents 3. In addition, the bulk density and pore size of the porous body constituting the sustained-release drug can be easily adjusted without use of a highly toxic solvent.

In the sustained-release drug produced by the method for producing a sustained-release drug of the present invention, the medicinal ingredient is supported on, adhered to, or contained in the wall of the porous body containing the bioabsorbable polymer.

Adjustment of the type and the mixing ratio of the one or two or more common solvents 3 has been confirmed to change the particle size of the medicinal ingredient exposed on the wall of the porous body. Presumably, there is a relation between the particle size of the medicinal ingredient exposed on the wall of the porous body and the releasing rate, and thus the releasing rate can be controlled by adjusting the type and mixing ratio of the one or two or more common solvents 3.

In the sustained-release drug, the lower limit of the average particle size of the medicinal ingredient exposed on the wall of the porous body is preferably 10 nm and the upper limit thereof is preferably 1000 μm. When the average particle size of the medicinal ingredient exposed on the wall of the porous body is within this range, a suitable releasing rate can be obtained. The lower limit of the average particle size of the medicinal ingredient exposed on the wall of the porous body is more preferably 30 nm, and the upper limit thereof is more preferably 900 μm. The lower limit is still more preferably 50 nm and the upper limit is still more preferably 800 μm.

The present invention is also directed to a sustained-release drug containing a medicinal ingredient supported on, adhered to, or contained in a wall of a porous body containing a bioabsorbable polymer, wherein the medicinal ingredient exposed on the wall of the porous body has an average particle size of 10 nm or larger and 1000 μm or smaller.

### Advantageous Effects of Invention

The present invention can provide a method for producing a sustained-release drug which includes allowing a medicinal ingredient to be supported on a porous body containing a bioabsorbable polymer and which can control the releasing rate of the medicinal ingredient. The present invention can also provide a sustained-release drug.

### BRIEF DESCRIPTION OF DRAWINGS

FIG. 1 is a scanning electron micrograph of a cross section of a sustained-release drug obtained in Example 1.

FIG. 2 is a scanning electron micrograph of a cross section of a sustained-release drug obtained in Example 2.

FIG. 3 is a scanning electron micrograph of a cross section of a sustained-release drug obtained in Example 3.

FIG. 4 shows curves representing the cumulative amounts of a medicinal ingredient (heparin) released from the sustained-release drugs obtained in Examples 1 to 3.

FIG. 5 is a scanning electron micrograph of a cross section of a sustained-release drug obtained in Example 4.

FIG. 6 shows weight loss curves obtained by heating the sustained-release drug obtained in Example 4.

### DESCRIPTION OF EMBODIMENTS

The embodiments of the present invention are described in more detail with reference to examples. The present invention however is not limited to these examples.

### Example 1

At a room temperature of 25° C., 0.25 g of a L-lactide-ε-caprolactone copolymer (molar ratio: 50:50, weight average molecular weight: 150000) was mixed with a mixed solution containing: 0.3 mL of water as the solvent 1 having a medicinal ingredient heparin (Wako Pure Chemical Industries, Ltd., heparin sodium, JIS guaranteed reagent) dissolved therein at a concentration of 7200 units/mL; 2.0 mL of methyl ethyl ketone as the solvent 2; and 1.0 mL of a 1:1 (volume ratio) mixture of acetone (common solvent 3-1) and alcohol (common solvent 3-2) (the alcohol was 100% ethanol). Thus, a non-uniform solution not dissolving the L-lactide-ε-caprolactone copolymer was obtained. The heparin in the non-uniform solution did not precipitate and formed stable micelles.

Subsequently, the obtained non-uniform solution was put in a glass tube having a diameter of 3.3 mm and heated at 55° C. to give a solution containing the heparin uniformly dispersed therein and the L-lactide-ε-caprolactone copolymer dissolved therein.

The obtained uniform solution was then cooled to 4° C. or −24° C. in a freezer to precipitate a porous body containing the L-lactide-ε-caprolactone copolymer and heparin.

The obtained porous body was immersed in an ethanol bath (50 mL) at 4° C. or −24° C. for 12 hours, and then immersed in 50 mL of t-butyl alcohol at 25° C. for 12 hours for washing.

Thereafter, the porous body was freeze-dried at −40° C. to give a cylindrical sustained-release drug having a diameter of 3.0 mm and a height of 15 mm.

FIG. 1 shows an electron micrograph of a cross section of the obtained sustained-release drug taken with a scanning electron microscope (Hitachi High-Technologies Corporation, TM-1000) at a magnification of 10000 times.

FIG. 1 shows that the obtained sustained-release drug had the medicinal ingredient (heparin) supported on, adhered to, or contained in the wall of the porous body containing the bioabsorbable polymer. The particle size of the medicinal ingredient (heparin) exposed on the porous body was measured. The average particle size was about 430 nm.

### Example 2

A sustained-release drug was obtained as in Example 1 except that the common solvent 3 used was 1.0 mL of a 1:1 (volume ratio) mixture of acetone (common solvent 3-1) and alcohol (common solvent 3-2) (the alcohol was a 90:10 (volume ratio) mixed solution of ethanol and propanol). In the obtained non-uniform solution, heparin did not precipitate and formed stable micelles.

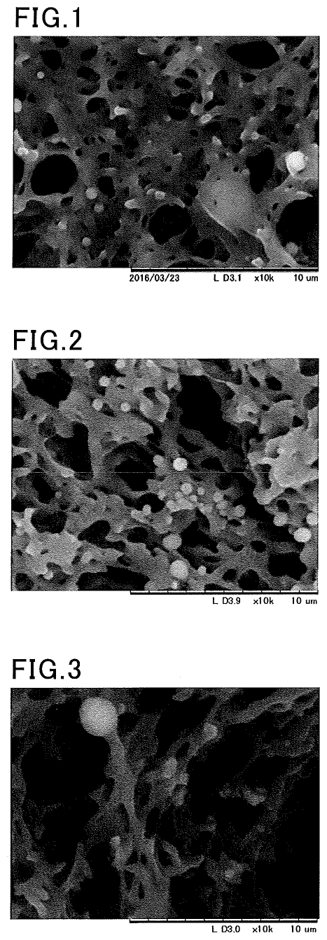

FIG. 2 shows an electron micrograph of a cross section of the obtained sustained-release drug taken with a scanning electron microscope (Hitachi High-Technologies Corporation, TM-1000) at a magnification of 10000 times.

FIG. 2 shows that the obtained sustained-release drug had the medicinal ingredient (heparin) supported on, adhered to, or contained in the wall of the porous body containing the bioabsorbable polymer. The particle size of the medicinal ingredient (heparin) exposed on the porous body was measured. The average particle size was about 780 nm.

### Example 3

A sustained-release drug was obtained as in Example 1 except that the common solvent 3 used was 1.0 mL of a 1:1 (volume ratio) mixture of acetone (common solvent 3-1) and alcohol (common solvent 3-2) (the alcohol was a 60:40 (volume ratio) mixed solution of ethanol and propanol).

In the obtained non-uniform solution, heparin did not precipitate and formed stable micelles.

FIG. 3 shows an electron micrograph of a cross section of the obtained sustained-release drug taken with a scanning electron microscope (Hitachi High-Technologies Corporation, TM-1000) at a magnification of 10000 times.

FIG. 3 shows that the obtained sustained-release drug had the medicinal ingredient (heparin) supported on, adhered to, or contained in the wall of the porous body containing the bioabsorbable polymer. The particle size of the medicinal ingredient (heparin) exposed on the porous body was measured. The average particle size was about 1300 nm.

### (Evaluation)

The heparin releasing rate of the sustained-release drugs obtained in Examples 1 to 3 was evaluated by the following method.

With incubation at 37° C., the amount of heparin released was measured by absorptiometry using toluidine blue (see Wollin, A. et al., Thromb. Res., vol. 2,377 (1973)).

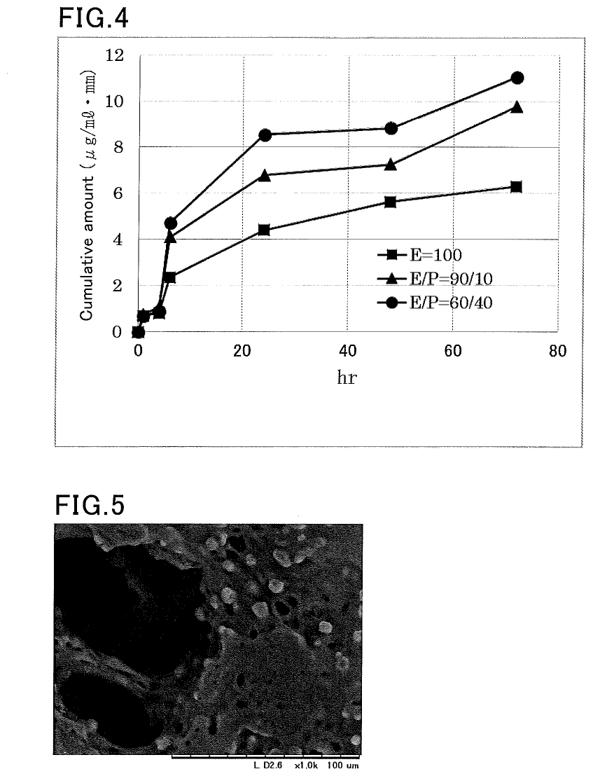

FIG. 4 shows curves representing the cumulative amounts of the medicinal ingredient (heparin) released from the obtained sustained-release drugs.

FIG. 4 shows that the releasing rate of the medicinal ingredient of the resulting sustained-release drug can be controlled by changing the composition of the common solvent 3 (composition of the alcohol as the common solvent 3-2).

In FIG. 4, ethanol is denoted by E and propanol is denoted by P.

### Example 4

At a room temperature of 25° C., 0.25 g of a L-lactide-ε-caprolactone copolymer (molar ratio: 50:50) and 0.22 g of a medicinal ingredient L-menthol (Wako Pure Chemical Industries, Ltd., JIS guaranteed reagent) were dissolved in 1.75 mL of methyl ethyl ketone as the solvent 2. Subsequently, to the obtained solution was added a mixed solution containing 1.2 mL of a 1:1 (volume ratio) mixture of acetone (common solvent 3-1) and ethanol (common solvent 3-2) and 0.25 mL of water as the solvent 1 with heating at 60° C. Thus, a uniform solution having the L-lactide-ε-caprolactone copolymer dissolved therein was obtained.

Subsequently, the obtained solution was cooled to 4° C. or −24° C. in a freezer to precipitate a porous body containing the L-lactide-ε-caprolactone copolymer and the L-menthol.

The obtained porous body was immersed in a water bath (50 mL) at 4° C. for 12 hours twice for washing.

Thereafter, the porous body was air-dried for 24 hours to give a cylindrical sustained-release drug having a diameter of 3.0 mm and a height of 15 mm.

FIG. 5 shows an electron micrograph of a cross section of the obtained sustained-release drug taken with a scanning electron microscope (Hitachi High-Technologies Corporation, TM-1000) at a magnification of 10000 times.

FIG. 5 shows that the obtained sustained-release drug had the medicinal ingredient (L-menthol) supported on, adhered to, or contained in the wall of the porous body containing the bioabsorbable polymer. The particle size of the medicinal ingredient (heparin) exposed on the porous body was measured. The average particle size was about 640 μm.

### (Evaluation)

The sustained release of the sustained-release drug obtained in Example 4 was evaluated by the following method.

Weight loss curves were drawn from TG curves obtained using a thermogravimetry/differential thermal analysis (TG/DTA) apparatus (PerkinElmer, STA 6000) under the following conditions: conditions a) the sustained-release drug is heated from 30° C. to 40° C. at 5° C./min, followed by keeping the drug isothermally at 40° C. for 30 minutes; and conditions b) the sustained-release drug is heated from 30° C. to 50° C. at 5° C./min, followed by keeping the drug isothermally at 50° C. for 30 minutes.

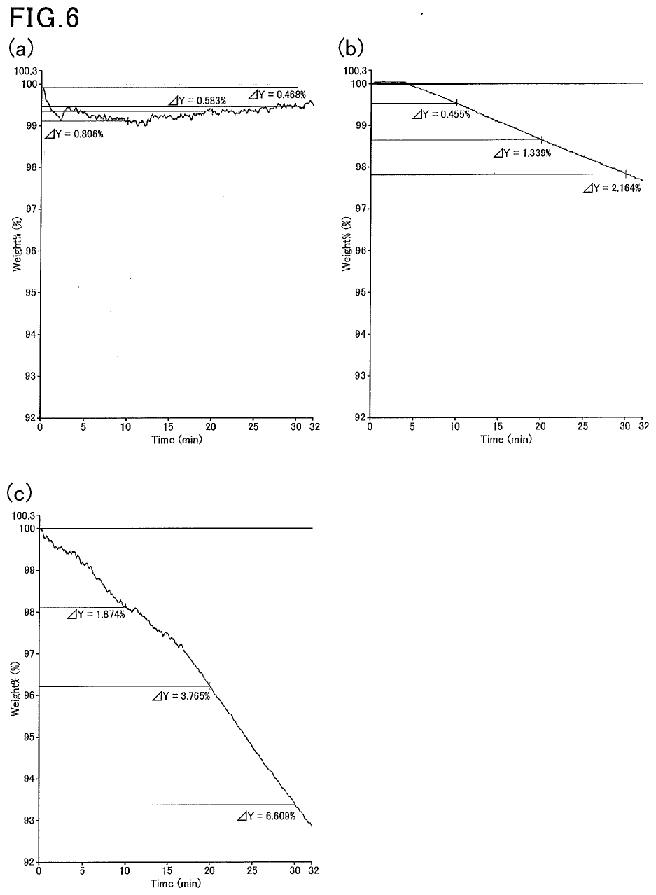

FIG. 6(a) (conditions a) and FIG. 6(b) (conditions b) show the obtained weight loss curves. For comparison, FIG. 6(c) shows a weight loss curve drawn from a TG curve obtained with L-menthol alone under conditions a) the sustained-release drug is heated from 30° C. to 40° C. at 5° C./min, followed by keeping the drug isothermally at 40° C. for 30 minutes.

FIG. 6 shows that while L-menthol alone exhibited significant volatility under conditions a), the sustained-release drug obtained in Example 4 hardly released the medicinal ingredient under conditions a) and moderately released it under conditions b).

### INDUSTRIAL APPLICABILITY

The present invention can provide a method for producing a sustained-release drug which includes allowing a medicinal ingredient to be supported on a porous body containing a bioabsorbable polymer and which can control the releasing rate of the medicinal ingredient. The present invention can also provide a sustained-release drug.

1. A method for producing a sustained-release drug, comprising:
a solution preparing step of preparing a medicinal ingredient-bioabsorbable polymer solution having a medicinal ingredient uniformly dispersed therein and a bioabsorbable polymer dissolved therein, using the bioabsorbable polymer, the medicinal ingredient, a solvent 1 that is a poor solvent having a lower solvency for the bioabsorbable polymer, a solvent 2 that is a good solvent having a higher solvency for the bioabsorbable polymer and is incompatible with the solvent 1, and a common solvent 3 compatible with the solvent 1 and the solvent 2;
a precipitating step of cooling the medicinal ingredient-bioabsorbable polymer solution to precipitate a porous body containing the bioabsorbable polymer and the medicinal ingredient; and
a freeze-drying step of freeze-drying the porous body containing the bioabsorbable polymer and the medicinal ingredient to provide a sustained-release drug containing a porous body supporting the medicinal ingredient,
wherein one or two or more common solvents 3 are used, and a releasing rate of the medicinal ingredient of the resulting sustained-release drug is controlled by adjusting a type and a mixing ratio of the one or two or more common solvents 3.

2. A sustained-release drug comprising
a medicinal ingredient supported on, adhered to, or contained in a wall of a porous body containing a bioabsorbable polymer,
wherein the medicinal ingredient exposed on the wall of the porous body has an average particle size of 10 nm or larger and 1000 μm or smaller.
